(19) United States
(12) Patent Application Publication
(10) Pub. No.: US 2021/0379319 A1
(43) Pub. Date: Dec. 9, 2021

(54) WIRE HEATED TUBE FOR RESPIRATORY APPARATUS

(71) Applicant: RESMED PTY LTD, Bella Vista, New South Wales (AU)

(72) Inventor: Andrew CHAN, Sydney (AU)

(21) Appl. No.: 17/285,834

(22) PCT Filed: Oct. 11, 2019
(86) PCT No.: PCT/IB2019/058706

§ 371 Date: Apr. 15, 2021

Related U.S. Application Data
(60) Provisional application No. 62/745,799, filed on Oct. 15, 2018.

(51) Int. Cl. A61M 16/10; A61M 16/16; A61M 16/08; A61M 16/06; A61M 16/00

(52) U.S. Cl. CPC A61M 16/1095; A61M 16/16; A61M 16/0816; A61M 16/0875; A61M 16/06; A61M 2016/0036; A61M 2205/3368; A61M 2205/3653; A61M 2205/14; A61M 2205/3317; A61M 2205/3327; A61M 16/022

(57) ABSTRACT

An air delivery conduit for use with an apparatus to deliver a supply of pressurized breathable air includes a tube, a first wire, a second wire, a first cuff, and a second cuff. The first wire extends at least partially between a first end and a second end of the tube. The first wire has a first diameter. The second wire extends at least partially between the first end and the second end. The second wire has a second diameter different than the first diameter. The first cuff is coupled to the first end of the tube. The second cuff is coupled to the second end of the tube and includes a thermistor connected to the first wire and a fixture that protrudes from an internal surface of the second cuff into a flow path of the supply of pressurized breathable air. The thermistor is enclosed within the fixture.

### CROSS-REFERENCE TO RELATED APPLICATIONS

This application claims the benefit of U.S. Provisional Patent Application No. 62/745,799, filed Oct. 15, 2018, which is incorporated herein by reference in its entirety.

This application incorporates the teachings of U.S. Pat. No. 9,572,949, filed Jan. 31, 2014 and U.S. Pat. No. 9,903,371, filed Aug. 28, 2009 (appended hererto) as if set forth in their entireties herein.

### FIELD

The present technology relates to heated air delivery conduits used in breathing appartatus, including for example, invasive and non-invasive ventilation, Continuous Positive Airway Pressure (CPAP), Bi-Level therapy and treatment for sleep disordered breathing (SDB) conditions such as Obstructive Sleep Apnea (OSA), and for various other respiratory disorders and diseases.

### BACKGROUND

Respiratory apparatus commonly have the ability to alter the humidity of the breathable gas in order to reduce drying of the patient's airway and consequent patient discomfort and associated complications. The use of a humidifier placed between the flow generator and the patient mask, produces humidified gas that minimizes drying of the nasal mucosa and increases patient airway comfort. In addition in cooler climates, warm air applied generally to the face area in and about the mask, as may occur inadvertently by a leak, is more comfortable than cold air.

The humidified air may cool on its path along the conduit from the humidifier to the patient, leading to the phenomenon of “rain-out”, or condensation, forming on the inside of the conduit. To counter this, it is known to additionally heat the gas being supplied to the patient by means of a heated wire circuit incorporated into the wall of the wire heated tube.

### BRIEF SUMMARY OF THE TECHNOLOGY

According to one aspect, an air delivery conduit includes a tube, a first wire and a second wire in the tube, and a thermistor connected to the first wire. The first wire includes a first diameter and the second wire includes a second diameter that is different from the first diameter.

In some aspects, a third wire is disposed in the tube and includes a third diameter that is different from the first diameter.

In some aspects, a fourth wire is disposed in the tube and includes a fourth diameter that is the same as the first diameter.

According to one aspect, a control system for a heated conduit includes a sensing circuit configured to indicate the temperature of a sensor positioned in the heated conduit. The sensing circuit includes a first wire with a first diameter and a second wire with a second diameter that is different than the first diameter.

According to one aspect, a sensing circuit for a heating conduit includes a sensing wire and a heating wire coupled to a heating circuit for the heated conduit. The sensing circuit also includes a temperature sensor coupled to the sensing wire and configured to measure the temperature of the heated conduit. The sensing wire has a first diameter and the heating wire has a second diameter that is different from the first diameter.

In some aspects, a second heating wire is coupled to the heating circuit and includes a third diameter that is different from the first diameter.

According to one aspect, an air delivery conduit for use with an apparatus to deliver a supply of pressurized breathable air to a patient includes a tube, a first wire, a second wire, a first cuff, and a second cuff The tube has a first end and a second end. The first wire extends at least partially between the first end and the second end. The first wire has a first diameter. The second wire extends at least partially between the first end and the second end. The second wire has a second diameter that is different than the first diameter. The first cuff is coupled to the first end of the tube and includes an electrical connector that is connected to the first wire and the second wire to provide an electrical connection with the apparatus. The second cuff is coupled to the second end of the tube. The second cuff includes a thermistor connected to the first wire, and a fixture that protrudes from an internal surface of the second cuff into a flow path of the supply of pressurized breathable air flowing through the second cuff The thermistor is enclosed within the fixture.

In some aspects, the tube has a spiral ribbing, and the first wire and the second wire are positioned within the spiral ribbing.

In some aspects, the electrical connector includes a first terminal that corresponds with the first wire and a second terminal that corresponds with the second wire. The first terminal and the second terminal are configured to receive contacts of the apparatus.

In some aspects, the second cuff further includes a first end portion with the internal surface fixed to an external surface of the tube, and a second end portion comprising an elastomeric material to frictionally engage an exterior surface of a tubular connector.

In some aspects, the first wire and the second wire are electrically connected to one another.

In some aspects, the second wire is a heating wire and is made of a low ohmic value resistors to apply heat to the tube.

In some aspects, a third wire extends at least partially between the first end and the second end. The third wire having a third diameter different than the first diameter.

In some aspects, the third wire is a heating wire and is made of a low ohmic value resistors to apply heat to the tube

In some aspects, the third wire is electrically connected to the second wire.

In some aspects, the third wire is electrically connected to the first wire.

In some aspects, the third wire is connected to ground.

In some aspects, the first wire monitors the temperature of the air proximate to the second cuff and detects any imbalance between the bridge formed by the second wire and the third wire.

In some aspects, the third diameter is equal to the second diameter.

In some aspects, a fourth wire extends at least partially between the first end and the second end. The fourth wire having a fourth diameter different than the second diameter.

In some aspects, the fourth wire is a sensing wire and is electrically connected to the thermistor and the first wire.

In some aspects, the fourth wire is included in a circuit distinct from the first wire.

In some aspects, the fourth wire is connected to ground.

In some aspects, the fourth diameter is equal to the first diameter.

In some aspects, the second diameter is larger than the first diameter.

In some aspects, the first diameter is equivalent to a gauge of 29 American Wire Gauge (AWG).

In some aspects, the second diameter is equivalent to a gauge of 31 AWG.

In some aspects, the tube, the first wire, and the second wire are flexible, and the first diameter increases the overall flexibility of the tube as compared to the second diameter.

According to one aspect, an air delivery conduit for use with an apparatus to deliver a supply of pressurized breathable air to a patient includes a tube, a first wire, a second wire, a third wire, a first cuff, and a second cuff The tube has a first end and a second end. The first wire extends at least partially between the first end and the second end. The first wire has a first diameter. The second wire extends at least partially between the first end and the second end. The second wire has a second diameter different than the first diameter. The third wire extends at least partially between the first end and the second end. The third wire has a third diameter different than the first diameter. The first cuff is coupled to the first end of the tube and includes an electrical connector connected to the first wire and the second wire to provide an electrical connection with the apparatus. A second cuff is coupled to the second end of the tube and includes a thermistor connected to the first wire.

In some aspects, a fixture protrudes from an internal surface of the second cuff into a flow path of the supply of pressurized breathable air flowing through the second cuff The thermistor is enclosed within the fixture.

In some aspects, the tube has a spiral ribbing, the first wire, the second wire, and the third wire are positioned within the spiral ribbing.

In some aspects, the electrical connector includes a first terminal that corresponds with the first wire, second terminal that corresponds to the second wire, and a third terminal that corresponds with the third wire. The first terminal, the second terminal, and the third terminal are configured to receive contacts of the apparatus.

In some aspects, the first wire, the second wire, and the third wire are electrically connected to one another.

In some aspects, the second wire and the third wire are heating wires and are made of a low ohmic value resistors to apply heat to the tube.

In some aspects, the third wire is connected to ground.

In some aspects, the first wire monitors the temperature of the air proximate to the second cuff and detects any imbalance between the bridge formed by the second wire and the third wire.

In some aspects, the third diameter is equal to the second diameter.

In some aspects, the first diameter is equivalent to a gauge of 29 American Wire Gauge (AWG).

In some aspects, the second diameter is equivalent to a gauge of 31 AWG.

According to one aspect, an air delivery conduit for use with an apparatus to deliver a supply of pressurized breathable air to a patient includes a tube, a first wire, a second wire, a third wire, a fourth wire, a thermistor, and a fixture. The tube has a first end and a second end. The first wire extends at least partially between the first end and the second end. The first wire has a first diameter. The second wire extends at least partially between the first end and the second end. The second wire has a second diameter that is different than the first diameter. The third wire extends at least partially between the first end and the second end. The third wire has a third diameter that is different than the first diameter. The fourth wire extends at least partially between the first end and the second end. The fourth wire has a fourth diameter different than the second diameter. The thermistor is connected to the first wire. The fixture protrudes from an internal surface of the tube into a flow path of the supply of pressurized breathable air flowing through the tube. The thermistor is enclosed within the fixture.

In some aspects, a cuff is coupled to the second end. The cuff includes the internal surface, and the fixture protrudes from the cuff.

In some aspects, the first wire and the fourth wire form a sensing circuit, and the second wire and the third wire form a heating circuit that is distinct from the sensing circuit.

In some aspects, the third wire and the fourth wire are connected to ground.

In some aspects, the first diameter is equal to the fourth diameter.

In some aspects, the second diameter is equal to the third diameter.

In some aspects, the first diameter is equivalent to a gauge of 29 American Wire Gauge (AWG).

In some aspects, the second diameter is equivalent to a gauge of 31 AWG.

### BRIEF DESCRIPTION OF THE DRAWINGS

Sample embodiments will be described with reference to the accompanying drawings, in which:

FIG. 1 schematically depicts a PAP system according to a sample embodiment;

FIG. 2 schematically depicts a PAP system according to another sample embodiment;

FIG. 3 schematically depicts a PAP system according to another sample embodiment;

FIG. 4 schematically depicts of a PAP system including a flow generator and humidifier according to a sample embodiment;

FIGS. 5-7 schematically depict the humidifier of FIG. 4;

FIG. 8 schematically depicts a heated tube according to a sample embodiment;

FIGS. 9-13 schematically depict a connector, or cuff, of the tube of FIG. 8 at an end of the tube configured to be connected to a humidifier;

FIG. 14 schematically depicts the end of the tube of FIGS. 9-13 connected to the humidifier of FIGS. 5-7;

FIG. 15 schematically depicts an end of the tube of FIG. 8 connected to a patient interface;

FIGS. 16 and 17 schematically depict a connector, or cuff, of the end of the tube of FIG. 8 configured to be connected to a patient interface;

FIG. 18 schematically depicts a wiring configuration for the heated tube of FIG. 8;

FIG. 19 schematically depicts a sample embodiment of an algorithm for controlling the heated tube;

FIG. 20 schematically depicts an alternate wiring configuration for a heated tube;

FIG. 21 schematically depicts another alternate wiring configuration for a heated tube; and

FIG. 22 schematically depicts a circuit according to another sample embodiment that senses temperature at the patient interface and provides active over temperature protection.

### DETAILED DESCRIPTION

### PAP System

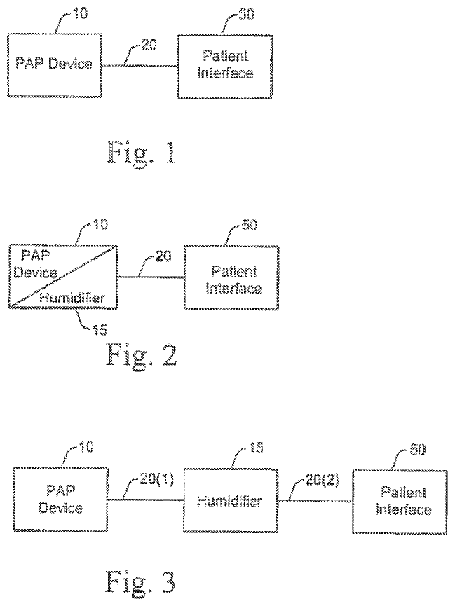

As schematically shown in FIG. 1, a Positive Airway Pressure (PAP) system, for example a Continuous Positive Airway Pressure (CPAP) system, generally includes a PAP device (or PAP system or respiratory apparatus) 10, an air delivery conduit 20 (also referred to as a tube or tubing), and a patient interface 50. In use, the PAP device 10 generates a supply of pressurized air that is delivered to the patient via an air delivery conduit 20 that includes one end coupled to the outlet of the PAP device 10 and an opposite end coupled to the inlet of the patient interface 50. The patient interface comfortably engages the patient's face and provides a seal. The patient interface or mask may have any suitable configuration as is known in the art, e.g., full-face mask, nasal mask, oro-nasal mask, mouth mask, nasal prongs, etc. Also, headgear may be utilized to comfortably support the patient interface in a desired position on the patient's face.

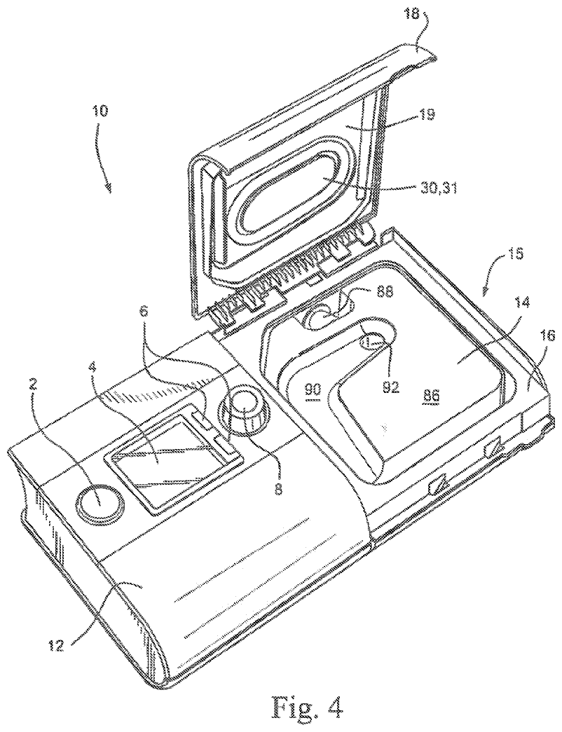

In embodiments, a humidifier may be incorporated or integrated into the PAP device or otherwise provided downstream of the PAP device. In such embodiments, the air delivery conduit 20 may be provided between the patient interface 50 and the outlet of the humidifier 15 as schematically shown in FIG. 2.

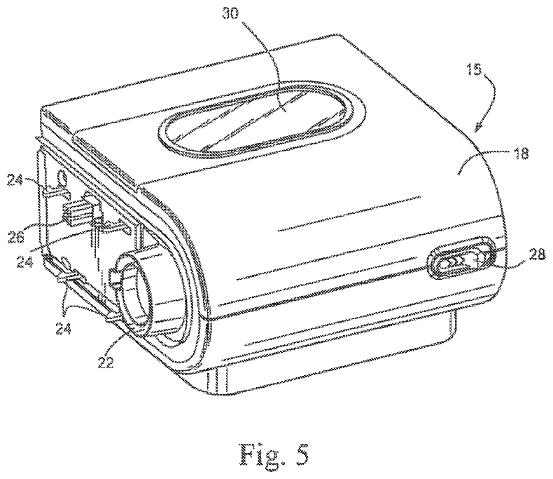

It should be appreciated that the air delivery conduit may be provided along the air delivery path in other suitable manners. For example, as schematically shown in FIG. 3, the humidifier 15 may be a separate component from the PAP device 10 so that an air delivery conduit 20(1) is placed between the PAP device 10 and the humidifier 15 and another air delivery conduit 20(2) is placed between the humidifier 15 and the patient interface 50.

Generally, a heated humidifier is used to provide sufficient humidity and temperature to the air so that the patient will be comfortable. In such embodiment, the air delivery conduit may be heated to heat the gas and prevent “rain-out” or condensation forming on the inside of the conduit as the gas is supplied to the patient. In this arrangement, the air delivery conduit may include one or more wires or sensors associated with heating.

As described below, each end of the air delivery conduit includes a cuff structured to attach the tube to the patient interface, PAP device, and/or humidifier. The cuffs differ for non-heated tubes and heated tubes, e.g., cuffs for heated tubes accommodate sensors or electronics/wiring associated with heating.

While the cuff is described as being implemented into a CPAP system of the type described above, it may be implemented into other tubing arrangements for conveying gas or liquid. That is, the CPAP system is merely exemplary, and aspects of the present invention may be incorporated into other suitable arrangements.

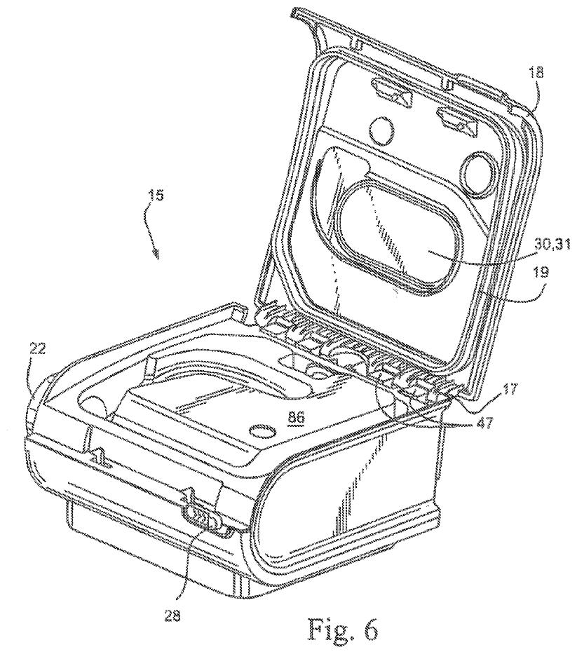

Referring to FIGS. 4-7, a PAP system 10 according to a sample embodiment comprises a flow generator, or blower, 12 and a humidifier 15. The flow generator 12 is configured to generate a flow of breathable gas having a pressure of, for example, about 2-30 cm H₂O. The flow generator comprises a power button 2 to turn the PAP system on and off. A display 4 is provided to display interactive menus and information regarding the operation of the PAP system to the user or operator. The user or operator may select menus and/or information through inputs 6, which may be, for example, buttons or keys. A push button dial 8 may also allow the user or operator to select information and/or menus. The inputs 6 and the push button dial 8 may be used together to select information and/or menus. For example, one or both of the inputs 6 may be pressed and the dial 8 may be rotated to display desired information or menu on the display 4 and the dial 8 may then be pressed to select particular information to be displayed or a particular mode of operation of the PAP system.

The humidifier 15 comprises a humidifier chamber 16 and a lid 18 which is pivotable between an open and a closed position. A water chamber, or tub, 14 is provided in the humidifier chamber 16 and is covered by the lid 18 when the lid 18 is in the closed position. A seal 19 is provided to the lid 18. The lid 18 includes a window 30 to allow visual inspection of the contents of the humidifier tub 14. The seal 19 includes an aperture 31 that corresponds to the position of the window 30 of the lid 18. In the closed position of the lid 18, the seal 19 contacts the tub 14 to ensure good thermal contact between a bottom of the tub 14 and a heating plate (not shown) provided in the bottom of the humidifier chamber 16 as disclosed, for example, in WO 2010/031126 A1. The tub 14 comprises a base, or bottom, that conducts heat from the heating plate to a supply of water provided in the tub 14. Such tubs are disclosed in WO 2010/031126 A1.

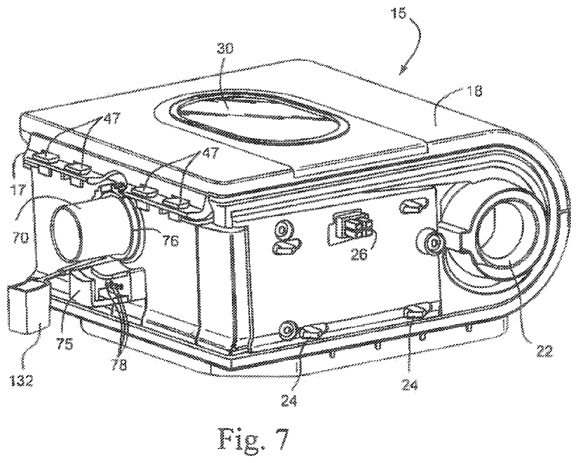

As shown in FIGS. 4 and 5, the humidifier 15 is connectable to the flow generator 12 by connectors, or latches, 24. The latches 24 may be, for example, spring biased latches that engage corresponding recesses (not shown) in the flow generator 12. An electrical connector 26 is provided to electrically connect the flow generator 12 to the humidifier tub 14. Electrical power may be provided from the flow generator 12 to the humidifier tub 14, although it should be appreciated that the humidifier may be provided with its own power source. Control signals may also be provided from the flow generator 12 to the humidifier tub 14 through the electrical connector 26.

As shown in FIG. 4, the tub 14 comprises a tub lid (or top) 86 that is configured to direct a flow of breathable gas generated by the flow generator 12 along a channel 90 in the tub lid 86 and through an outlet 92 of the channel 90 into the tub 14. The humidifier chamber 16 includes an air inlet 22 configured to receive the flow of breathable gas generated by the flow generator 12 when the humidifier 15 is connected to the flow generator 12 by the latches 24. The inlet 22 directs the flow into the channel 90 in the tub lid 86 of the humidifier tub 14. The flow is directed by the channel 90 to the outlet 92 into the humidifier tub 14. The tub 14 includes an outlet 88 for the humidified flow of breathable gas. A tube connector 70 (FIG. 7) is provided at a rear portion of the humidifier 15 in communication with the outlet 88. It should be appreciated that the tube connector 70 may be provided on a side, or the front, of the humidifier 15. The tube connector 70 is configured for connection to a hose, tube, or conduit to a tube that is configured to deliver the humidified flow to patient interface, e.g. a mask, as described in more detail herein.

It should be appreciated that the humidifier 15 may include its own control system, or controller, for example, a microprocessor provided on a printed circuit board (PCB). The PCB may be located in the wall of the humidifier chamber 16 and may include a light, e.g. an LED, to illuminate the contents of the tub 14 to permit visual inspection of the water level. It should also be appreciated that the flow generator 12 comprises a control system, or controller, that communicates with the controller of the humidifier 15 when the flow generator 12 and the humidifier 15 are electrically connected. It should be further appreciated that the flow generator and/or the humidifier may include a plurality of sensors, including for example, an ambient humidity sensor that may be configured to detect, for example, absolute ambient humidity and which may include an absolute humidity sensor or a temperature sensor to detect an ambient temperature and a relative humidity sensor to detect an relative humidity from which the ambient absolute humidity may be calculated. The plurality of sensors may also include, for example, an ambient pressure sensor to detect an ambient pressure, a flow sensor to detect a flow of breathable gas generated by the flow generator, and/or a temperature sensor to detect a temperature of a supply of water contained in the tub 14 of the humidifier 15 or the temperature of the heating plate of the humidifier 15. Such an arrangement is shown, for example, in U.S. Patent Application Publication 2009/0223514 A1. The PAP system 10 may be operated according to various control algorithms stored in the controller(s) of the flow generator 12 and/or the humidifier 15. Such control algorithms are disclosed in, for example, U.S. Patent Application Publication 2009/02223514 A1.

The humidifier 15 comprises the humidifier chamber 16 and the lid 18 which is pivotally connected to the humidifier chamber 16. As shown in FIG. 6, the lid 18 comprises a hinge portion 17 that is hinged to hinge portions 47 provided on the humidifier chamber 16. An opening member 28 is provided for releasing the lid 18 to allow the lid to be pivoted to the open position shown in FIGS. 4 and 6 as described in WO 2010/031126 A1.

Referring to FIG. 7, the humidifier comprises the tube connector 70 and a tube electrical connector 75. The tube connector 70 and the tube electrical connector 75 provide the ability to connect both a standard tube and a heated tube. As shown in FIG. 7, the tube electrical connector 75 comprises a plurality of contacts 78. Three contacts 78 are shown, although any number of contacts 78 (e.g., two, four, five, etc.) may comprise the plurality of contacts 78. The tube electrical connector 75 and the contacts 78 are provided separately from the tube connector 70. A heated tube having corresponding electrical connections, e.g. terminals, may be provided in a rotational snap fit with the tube electrical connector 75 as described in more detail below. This type of connection provides ease of connection and reduces the tolerance stack of the PAP system 10. A cover 132 may be connected to the back wall of the humidifier 15 to cover the tube connector 75 and the contacts 78 when a non-heated tube is connected to the tube connector 70. The cover 132 may be formed of a pliable rubber or other suitable flexible material. Alternatively the cover 132 may be a separate component, not attached to the humidifier that may be inserted over the tube electrical connector 75.

### Heated Tube/Conduit

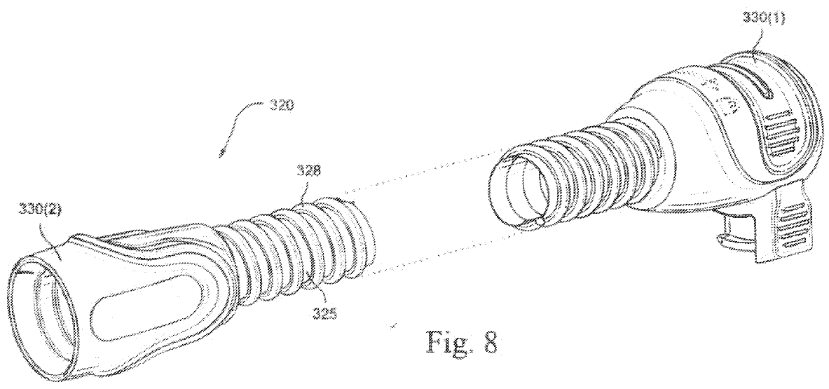

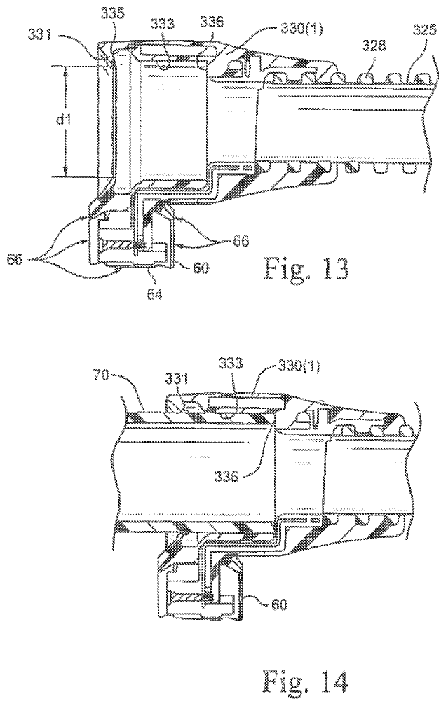

FIG. 8 illustrates an embodiment of a heated air delivery conduit or tube. The heated tube 320 comprises a flexible tube 325, a first connector, or cuff, 330(1) provided to one end of the tube 325 and configured and arranged to engage the tube connector 70 and the tube electrical connector of the humidifier 15, and a second cuff 330(2) provided to the opposite end of the tube 325 and configured and arranged to engage the inlet (e.g. a swivel elbow) of a patient interface 50, as shown in FIG. 15. The heated tube 320 may be, for example, as disclosed in U.S. Patent Application Publication 2010/0116272 A1.

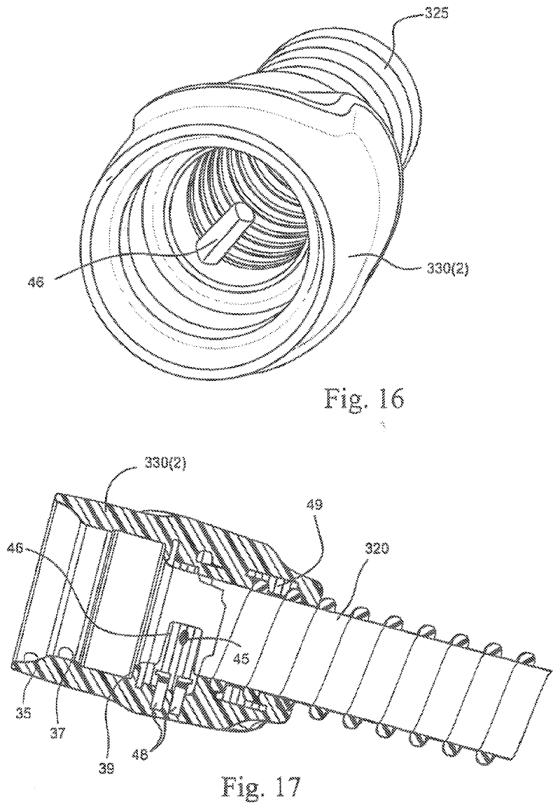

The tube 320 is structured to conduct heat along at least a portion of its length. For example, spiral ribbing 328 of the tube 325 may be structured to support three wires 504, 506, 508 (FIGS. 15 and 18). In addition, the heated tube 320 may be structured to support one or more sensing apparatus, e.g. a flow sensor and/or a temperature sensor, etc. Further details of such tubing are disclosed in U.S. Patent Application Publication 2008/0105257 A1.

In the illustrated embodiment, the cuffs 330(1), 330(2) are different from one another as described below. However, each cuff provides structure for attaching, sealing, and retaining the cuff to a respective connector, e.g., 22 mm ISO-taper connector.

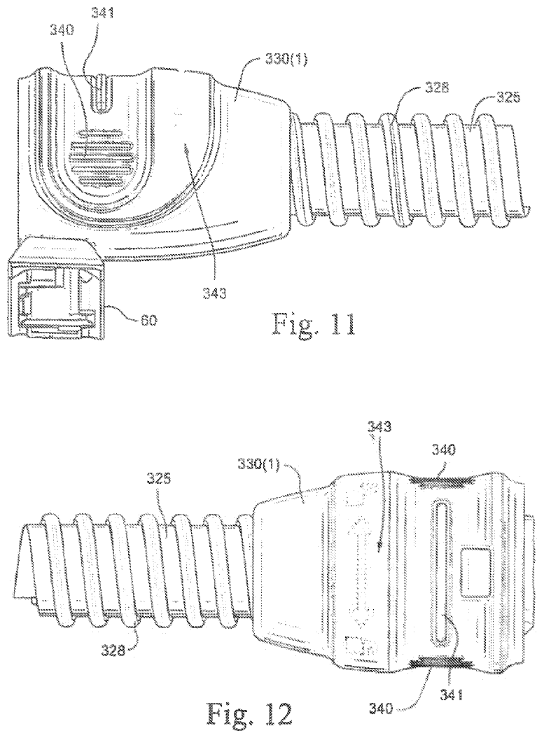

The opening of the cuff 330(1) includes a radial lip seal or sealing lip 331 along the interior surface thereof As shown in FIG. 13, the radial sealing lip 331, in its relaxed, undeformed shape, provides an internal diameter d1 that is smaller than the external diameter of the tube connector 70. For example, the internal diameter may be less than about 22 mm (e.g., about 19-21 mm or less) for use with a standard 22 mm connector. In use, as best shown in FIG. 14, the sealing lip 331 is structured to resiliently deform upon engagement with the tube connector 70 so as to provide a gas tight seal against the exterior surface of the tube connector 70. For example, the sealing lip 331 provides a flexible protrusion structured to resiliently deflect from a first position (FIG. 13) and into a second position (FIG. 14) within a cut-out 335.

As illustrated, the sealing lip 331 tapers outwardly towards the cuff opening to provide a sufficient lead in for aligning and engaging the cuff 330(1) with the tube connector 70.

The interior surface 333 axially inwardly from the sealing lip 331 provides an internal diameter that is substantially the same as the external diameter of the tube connector 70, e.g., about 22 mm for use with a standard 22 mm connector. A stop surface or flanged faced 336 within the cuff 330(1) provides a stop to prevent the tube connector 70 from inserting further into the cuff 330(1).

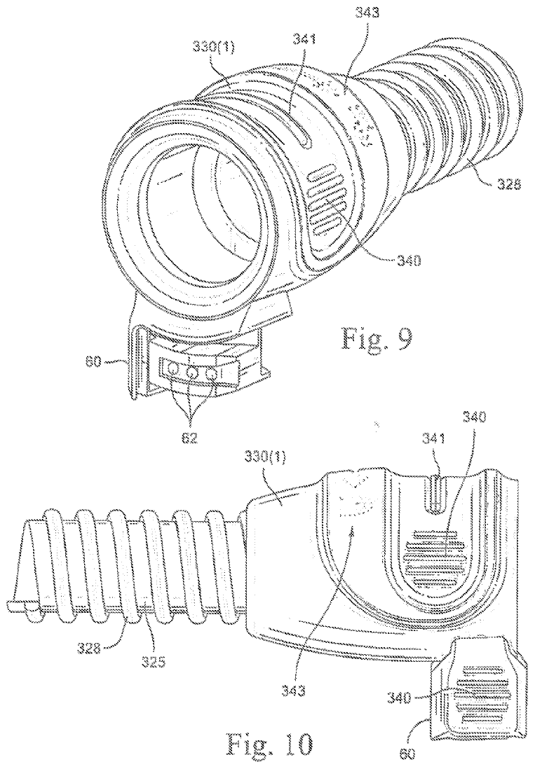

FIGS. 9-14 illustrate the cuff 330(1) structured for attachment to the humidifier 15. The cuff 330(1) includes an electrical connector 60 that is configured to provide an electrical connection with the humidifier 15 for operating the heating wires 504, 506, 508 (FIG. 15) provided to the tube 320. The electrical connector 60 includes terminals 62 that are configured to receive the contacts 78 of the tube electrical connector 75 of the humidifier 15 when the cuff 330(1) is connected to the tube connector 70 of the humidifier 15. The electrical connector 60 provides a retention function for the cuff 330(1). Retention is via a rotate-and-lock system to align the terminals 62 of the electrical connector 60 with the contacts 78 of the tube electrical connector 75 of the humidifier 15. The electrical connector 60 provides a heel 64 structured to be rotated into engagement with the tube electrical connector 75 such that the heel 64 locks into a cam or recess provided to the tube electrical connector 75 of the humidifier 15. When engaged, the heel 64 axially locks the cuff 330(1) into place. To release, the cuff 330(1) is rotated out of engagement with the tube electrical connector 75 to disengage the heel 64. As shown in FIG. 13, a seal 66 extends from the front, back, side, and bottom of the electrical connector 60 and seals against the tube electrical connector 75 of the humidifier 15 to prevent water spillage onto the electrical contacts 78 and the terminals 62.

In the illustrated embodiment, a number of terminals 62 is equal to the number of contacts 78 (i.e., there are three terminals 62 in the illustrated embodiment). In other embodiments, the number of terminals 62 may change in order to match the number of contacts 78 (e.g., the number of terminals 62 may be two, four, five, etc.). In still other embodiments, the number of terminals 62 and the number of contacts 78 may be unequal (e.g., there are greater or fewer terminals 62 than contacts 78).

The cuff 330(1) may comprise finger grips 340 along opposing sides thereof and along an edge of the electrical connector 60. The cuff 330(1) may also comprise an identifying strip 341 (e.g., orange strip) to identify the tube as a heated tube. A similar identifying strip may be provided to the user interface of the PAP system 10 and configured to illuminate or otherwise signal when the heated tube is operative, e.g., heating up, heated, etc. In addition, indicia and/or images 343 may be provided to the cuff 330(1) to indicate directions for locking and unlocking the cuff 330(1) with respect to the humidifier 15.

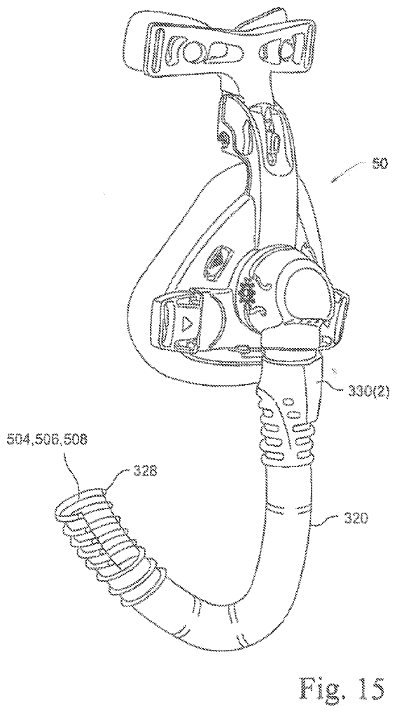

Referring to FIGS. 15-18, the cuff 330(2) at the opposite end of the heated tube 320 is configured for attachment to the patient interface (e.g. mask) 50. The cuff 330(2) comprises a sensor 45 located (e.g., molded into) within the rear portion of the cuff The cuff 330(2) includes a curved entry surface 35, a sealing and retention bead 37, and a stop surface 39 to aid connection of the heated tube 320 to the patient interface 50.

The sensor 45 is provided to a fixture 46 within the cuff. In the illustrated embodiment, the fixture 46 is wing-shaped (e.g. air-foil shaped) to optimize convective heat transfer over a range of flow rates, while minimizing noise or pressure drop. However, the fixture 46 may have other suitable shapes and/or textures. The cuff 330(2) may be formed by, for example, overmolding on a pre-block 49, or any method disclosed, for example, in U.S. Patent Application Publication 2008/0105257 A1, which is incorporated herein by reference in its entirety. The sensor 45 may be connected to the wires 504, 506, 508 in the heated tube 320 by a lead frame 48. The temperature sensed by the sensor 45 may be provided as a signal from the middle wire 504 through the lead frame 48 to a controller located in the humidifier 15 and/or the PAP system 10.

As shown in FIG. 18, the sensor 45 may take the form of a thermistor 410 formed of a Negative Temperature Coefficient (NTC) material. The middle wire 504 of the three wires 504, 506, 508 of the tube circuit 402 may be connected to the thermistor 410 and provide the temperature sensing signal to the controller. Two wires 506, 508 may be joined together at the lead frame 48 to complete the heating circuit. The third wire 504 provides a connection to the NTC thermistor which may be attached to the mid-point 507 of the heating circuit. The two heating wires 506, 508 may be low ohmic value resistors to apply heat to the tube wall and therefore to the air being delivered to the patient. The signal wire 504 may be fitted with the thermistor 410 located at the patient interface end of the heated tube 320. The signal wire 504 monitors the temperature of the air at the patient interface end of the heated tube and detects any imbalance between the bridge formed by the two heater wires 506, 508. The imbalance may be used to detect a fault condition, for example high impedance or an open circuit and low impedance or a short circuit.

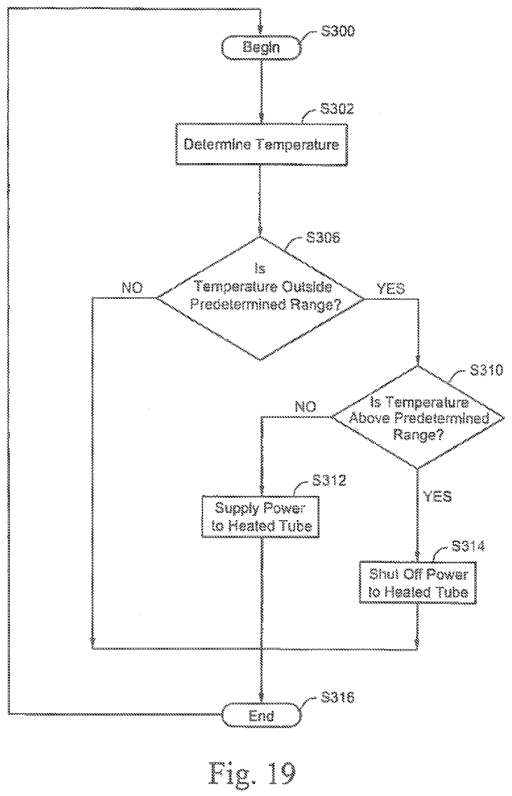

FIG. 20 is a schematic of an alternate embodiment of the three wire tube shown in FIG. 18. The three wires of the heated tube circuit 402a are arranged as described with regard to FIG. 18, but the middle wire 504a (wire 2, the sensing wire) has a different gauge than the two outer wires 506a, 508a (wires 1, 3, the heating wires). For example, the heating wires 506a, 508a may have a gauge of 31 AWG while the sensing wire 504a may have a gauge of 29 AWG, although any gauge of wire may be used. The middle wire 504a is connected to a thermistor 410a and provides the temperature sensing signal to the controller. The middle wire 504a is also connected to the heating wires 506a, 508a at a mid-point 507.

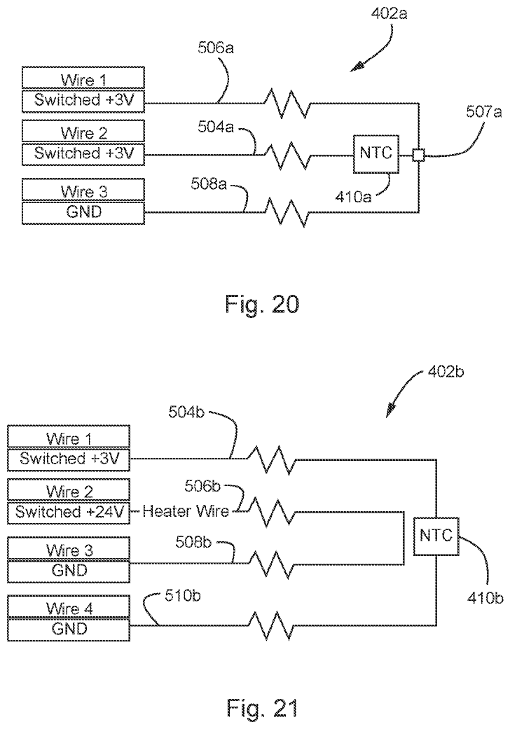

FIG. 21 is a schematic of an embodiment of the heated tube with a heated tube circuit 402b having four wires. In this embodiment two wires 506b, 508b (wires 2, 3) are joined together to form a heating circuit and two wires 504b, 510b (wires 1 and 4) are joined together to form a sensing circuit distinct from the heating circuit. Wires 1 and 4 504b, 510b may have a different gauge than wires 2 and 3 506b, 508b. For example, wires 2 and 3 506b, 508b (the heating wires) may have a gauge of 31 AWG while wires 1 and 4 504b, 510b (the sensing wires) may have a gauge of 29 AWG, although any gauge of wire may be used. Wires 1 and 4 504b, 510b are connected through a thermistor 410b which provides the temperature sensing signal to the controller.

The heated tube embodiments that utilize different gauge wires (e.g. as described with reference to FIGS. 20 and 21) may provide several benefits, both in clinical utility and manufacturability. By way of example, the use of thinner gauge wire increases the overall flexibility and decreases the overall weight of the tube as compared to similarly constructed tubes but which utilize the same gauge for each wire. This increase in flexibility and decrease in weight have clinical benefit to the patient as they can have an effect on comfort while receiving therapy. Additionally, the use of thinner gauge wire where current carrying requirements are lessened (e.g. sensing only wires) decreases the overall cost of manufacturing the tube as the metal volume in each tube is reduced.

### Heated Tube Control

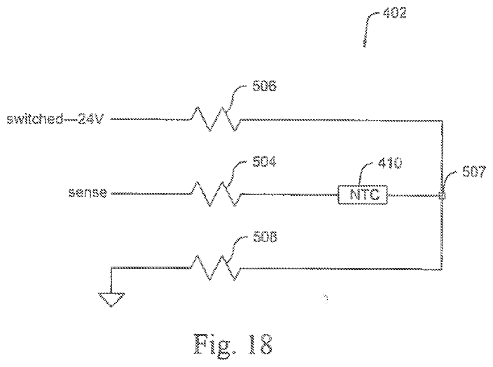

The heated tube 320 may be used to deliver the comfort of warm, humidified air and minimise condensation in the tubing. Referring to FIG. 19, an algorithm for controlling a heated tube is shown. The algorithm starts at S300 and determines the temperature sensed by a temperature sensor in the heated tube (e.g. thermistor 410) in S302. The algorithm proceeds to S306 and determines if the sensed temperature is outside a predetermined range. If the temperature of the heated tube is not outside the predetermined range (S306: No), the algorithm ends in S316. Conversely, if the temperature is outside the predetermined range (S306: Yes) the algorithm proceeds to S310 and it is determined if the temperature is above the predetermined range. If the temperature is below the predetermined range (S310: No), the algorithm proceeds to S312 and power is supplied to the heated tube. If the sensed temperature is above the predetermined range (S310: Yes), the algorithm proceeds to S314 shuts off power to the heated tube. After the completion of S312 or S314 the algorithm returns to the beginning in S300, thus providing temperature control for the heated tube.

The control of the heated tube may involve several considerations. One consideration is to measure and control the delivered air temperature in the heated tube system with a low cost tube assembly. Another consideration is, for safety, a failsafe mechanism may be provided to ensure the delivered air temperature does not exceed a safe temperature limit. Still another consideration is that it may be desirable to automatically identify whether the heated tube that is attached to the humidifier and/or flow generator has a 15 mm or 19 mm internal diameter. The pneumatic performance of the system may require compensation in the blower drive circuitry depending on which internal diameter tube is present.

According to another consideration, for safety, it is desirable to detect failures in the heated tube, such as high resistance hot spots in the wires or short circuits between the wires part way down the length of the tubing. A further consideration is that the heated tube may make both electrical and pneumatic connection to the humidifier in a simple attachment process.

Current heated tube systems do not directly regulate the temperature of the air delivered. They are implemented as open loop control of tube heating using a fixed power level. Although it may be possible to implement a thermal cut-out switch within the structure of the tube, these devices are relatively large and require additional circuit connections and mechanical mounting that add significant complexity to the tube.

### Heated Tube Control—Temperature Sensing with Active Over Temperature Protection

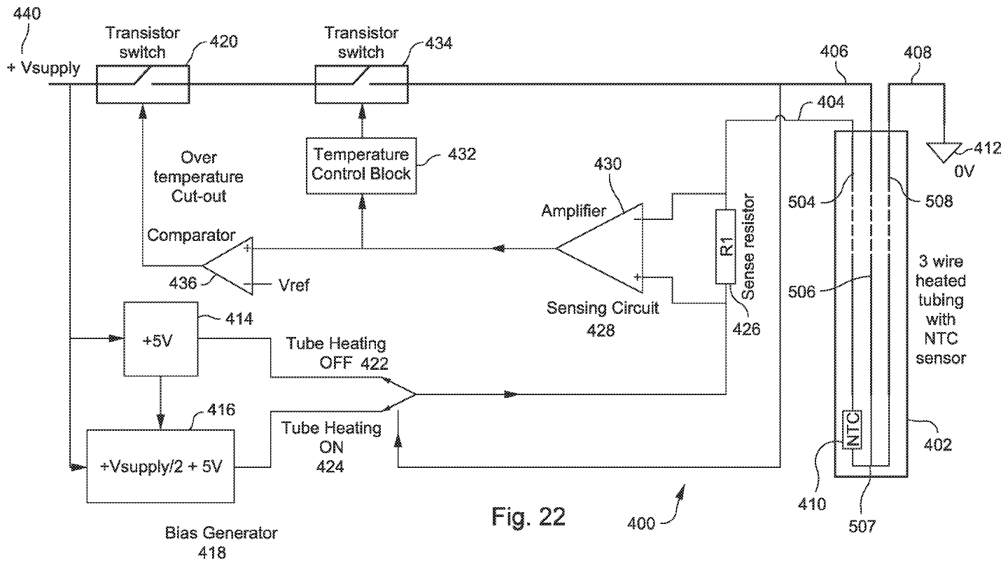

Referring to FIG. 22, a circuit configuration 400 according to a sample embodiment allows control of the tube air temperature using a sensor at the output (mask) end of the tube. The heated tube circuit 402 comprises the three wires 504,506, 508 and the temperature sensor, e.g. the NTC thermistor 410 that are located within the heated tube. The wires 404, 406, 408 are used in the sensing and control circuit to create a lower cost heating and sensing system with only three wires and are connected to the three wires 504, 506, 508 respectively. As shown in FIG. 18, the three wires 504, 506, 508 of the heated tube circuit 402 are connected to different components of the sensing and control circuit to provide a sensing wire 404, 504, a power supply wire 406 and a ground wire 408. In other embodiments of FIG. 22, the three wires 504a, 506a, 508a of the heated tube circuit 402a in FIG. 20 or the four wires 504b, 506b, 508b, 510b of the heated tube circuit 402b of FIG. 21 may be used in place of the heated tube circuit 402 of FIG. 18. The sensing and control circuits may be provided in a power supply and controller of the humidifier and/or flow generator. Such a power supply and controller is disclosed in, for example, U.S. Patent Application Publication 2008/0105257 A1. The complete sensing wire is formed of wires 404 and 504.

Referring again to FIG. 22, the circuit configuration 400 comprises a power supply 440, such as a 24V supply voltage, an over-temperature control circuit and a heating control circuit. The over-temperature control circuit comprises a first transistor switch 420 that is turned on when the temperature of the heated tube is below a predetermined temperature and turned off when the temperature is at or above the predetermined temperature. The predetermined temperature is set at a temperature to meet appropriate safety requirements of the heated tube, such as between 30° C. and 45° C., preferably 38° C. to 43° C. Comparator 436 controls the switching of the transistor switch 420. A reference voltage representing the predetermined temperature is compared to the voltage determined from an amplifier 430 from the sensing circuit to ensure the heated tube is not above or equal to the predetermined temperature.

Within the over-temperature control circuit is the heating control circuit which is designed to control the heating of the heated tube to obtain a desired temperature. The desired temperature may be set by the user or determined by the system. The heating control circuit switches the power supply 440 through the heated tube circuit 402 to a ground reference 412. Thus, the temperature sensor 410 moves between ground having 0V and half the supply voltage, e.g. 12V. Heating is supplied to the heated tube circuit 402 from power supply 440 through a second transistor switch 434. Transistor switch 434 is open and closed to turn heating on and off to the heated tube circuit 402 respectively. In one embodiment this transistor switch 434 is switched on and off very rapidly with changes in the duty cycle to control the heating of the tube. However, the switch 434 may be switched on to provide constant heating until a set temperature is reached and then turned off The temperature of the heated tube is sensed by the temperature sensor 410 and is transmitted through sense wire 404, 504 to sensing resistor 426 and sensing circuit 428 comprising amplifier 430. A bias generator circuit 418 provides the bias source voltage Vcc for the sensing circuit 428 so that the temperature of the heated tube is determined whether the tube is being heated or not. The bias generator circuit 418 generates a reference voltage that is either the Vcc bias source voltage 414, shown as 5V in this embodiment although other voltages may be used, when the tube heating is off via switch 422 or provides half the voltage supply plus the Vcc bias source voltage 416, i.e. 5V, when the tube heating is on via switch 424. Thus a constant voltage of Vcc bias source voltage is provided across the sensing circuit 428 irrespective of the state of the heated tube. The switching of the bias switches 422, 424 is controlled by the transistor switch 434 of the heating control circuit, such that when the transistor switch 434 is closed the tube heating ON switch 424 is active and when the transistor switch 434 is open the tube heating ON switch 424 is inactive. Thus, it is the voltage that is supplied to the heated tube circuit 402 that provides the bias switch.

The sensed temperature signal from the temperature sensor 410 is provided to amplifier 430 that produces a voltage that represents the heated tube temperature. The temperature control block 432 controls the opening and closing of switch 434 to modulate the power delivered to the heated tube circuit to maintain the desired temperature.

The temperature sensor 410 is held at a different circuit potential when the heater is active and when it is inactive. However, the sensor 410 should be continuously monitored to provide a failsafe against over temperature. A bias circuit 418 may be provided for continuous sensing. A bias generator circuit may provide the source voltage for the sensing circuit, a divider network comprising a resistor R1 and the NTC thermistor. This allows continuous temperature monitoring during both heating and idle states of the sensing and control system, and facilitates an active over temperature detection that is independent of the temperature control loop. Temperature sensing also remains active during the over temperature condition.

The circuit configuration may comprise a common ground referenced heating/sensing system with a supply voltage switching to the tube circuit for heating control. An alternative approach is to utilise the supply voltage as both the heating and sensing source voltage and control heating by switching to 0V the tube circuit.

### Alternative Bias Generator Arrangements

As described above the bias generator allows for a three wire or four wire heated tube system to provide temperature sensing during the active heating or ON cycle of the heating circuit as well as during the inactive or OFF cycle of the heating circuit. Temperature sensing remains active during at least a portion, such as at least 50%, at least 75% or at least 90% or during 100% of both the active (ON) heating cycle and the inactive (OFF) heating cycle. Thus the temperature sensing circuit may provide temperature sensing throughout use of the heated tube irrespective of the heating status of the system.

The heating tube circuit (e.g., 402, 402a, 402b) can be used with an alternate bias generator arrangement, for example as described in FIGS. 20A-22 and 27 and the accompanying description of U.S. Pat. No. 9,572,949 B2, which is incorporated herein by reference in its entirety.

1. An air delivery conduit for use with an apparatus to deliver a supply of pressurized breathable air to a patient, the air delivery conduit comprising:
a tube having a first end and a second end;
a first wire extending at least partially between the first end and the second end, the first wire having a first diameter;
a second wire extending at least partially between the first end and the second end, the second wire having a second diameter different than the first diameter;
a first cuff coupled to the first end of the tube and including an electrical connector connected to the first wire and the second wire to provide an electrical connection with the apparatus; and
a second cuff coupled to the second end of the tube and said second cuff comprising, a thermistor connected to the first wire, and
a fixture protruding from an internal surface of the second cuff into a flow path of the supply of pressurized breathable air flowing through the second cuff, the thermistor being enclosed within the fixture.

2. The air delivery conduit of claim 1, wherein the tube has a spiral ribbing, the first wire and the second wire are positioned within the spiral ribbing.

3. The air delivery conduit of claim 1, wherein the electrical connector includes a first terminal that corresponds with the first wire and a second terminal that corresponds with the second wire, the first terminal and the second terminal are configured to receive contacts of the apparatus.

4. The air delivery conduit of claim 1, wherein the second cuff further includes,
a first end portion having the internal surface fixed to an external surface of the tube; and
a second end portion comprising an elastomeric material to frictionally engage an exterior surface of a tubular connector.

5. The air delivery conduit of claim 1, wherein the first wire and the second wire are electrically connected to one another.

6. The air delivery conduit of claim 1, wherein the second wire is a heating wire and is made of a low ohmic value resistors to apply heat to the tube.

7. The air delivery conduit of claim 1 further comprising a third wire extending at least partially between the first end and the second end, the third wire having a third diameter different than the first diameter.

8. The air delivery conduit of claim 7, wherein the third wire is a heating wire and is made of a low ohmic value resistors to apply heat to the tube.

9. The air delivery conduit of claim 7, wherein the third wire is electrically connected to the second wire.

10. The air delivery conduit of claim 7, wherein the third wire is electrically connected to the first wire.

11. The air delivery conduit of claim 7, wherein the third wire is connected to ground.

12. The air delivery conduit of claim 7, wherein the first wire monitors a temperature of air proximate to the second cuff and detects any imbalance between a bridge formed by the second wire and the third wire.

13. The air delivery conduit of claim 7, wherein the third diameter is equal to the second diameter.

14. The air delivery conduit of claim 7, further comprising a fourth wire extending at least partially between the first end and the second end, the fourth wire having a fourth diameter different than the second diameter.

15. The air delivery conduit of claim 14, wherein the fourth wire is a sensing wire and is electrically connected to the thermistor and the first wire.

16. The air delivery conduit of claim 14, wherein the fourth wire is included in a circuit distinct from the first wire.

17. The air delivery conduit of claim 14, wherein the fourth wire is connected to ground.

18. The air delivery conduit of claim 14, wherein the fourth diameter is equal to the first diameter.

19. The air delivery conduit of claim 1, wherein the second diameter is larger than the first diameter.

20. The air delivery conduit of claim 1, wherein the first diameter is equivalent to a gauge of 29 American Wire Gauge (AWG).

21. The air delivery conduit of claim 1, wherein the second diameter is equivalent to a gauge of 31 AWG.

22. The air delivery conduit of claim 1, wherein the tube, the first wire, and the second wire are flexible, and the first diameter increases overall flexibility of the tube as compared to the second diameter.

23. An air delivery conduit for use with an apparatus to deliver a supply of pressurized breathable air to a patient, the air delivery conduit comprising:
a tube having a first end and a second end;
a first wire extending at least partially between the first end and the second end, the first wire having a first diameter;
a second wire extending at least partially between the first end and the second end, the second wire having a second diameter different than the first diameter;
a third wire extending at least partially between the first end and the second end, the third wire having a third diameter different than the first diameter;
a first cuff coupled to the first end of the tube and including an electrical connector connected to the first wire and the second wire to provide an electrical connection with the apparatus; and
a second cuff coupled to the second end of the tube and said second cuff comprising, a thermistor connected to the first wire.

24. The air delivery conduit of claim 23, further comprising a fixture protruding from an internal surface of the second cuff into a flow path of the supply of pressurized breathable air flowing through the second cuff, the thermistor being enclosed within the fixture.

25. The air delivery conduit of claim 23, wherein the tube has a spiral ribbing, the first wire, the second wire, and the third wire are positioned within the spiral ribbing.

26. The air delivery conduit of claim 23, wherein the electrical connector includes a first terminal that corresponds with the first wire, second terminal that corresponds to the second wire, and a third terminal that corresponds with the third wire, the first terminal, the second terminal, and the third terminal are configured to receive contacts of the apparatus.

27. The air delivery conduit of claim 23, wherein the first wire, the second wire, and the third wire are electrically connected to one another.

28. The air delivery conduit of claim 23, wherein the second wire and the third wire are heating wires and are made of a low ohmic value resistors to apply heat to the tube.

29. The air delivery conduit of claim 23, wherein the third wire is connected to ground.

30. The air delivery conduit of claim 23, wherein the first wire monitors a temperature of air proximate to the second cuff and detects any imbalance between a bridge formed by the second wire and the third wire.

31. The air delivery conduit of claim 23, wherein the third diameter is equal to the second diameter.

32. The air delivery conduit of claim 23, wherein the first diameter is equivalent to a gauge of 29 American Wire Gauge (AWG).

33. The air delivery conduit of claim 23, wherein the second diameter is equivalent to a gauge of 31 AWG.

34. An air delivery conduit for use with an apparatus to deliver a supply of pressurized breathable air to a patient, the air delivery conduit comprising:
a tube having a first end and a second end;
a first wire extending at least partially between the first end and the second end, the first wire having a first diameter;
a second wire extending at least partially between the first end and the second end, the second wire having a second diameter different than the first diameter;
a third wire extending at least partially between the first end and the second end, the third wire having a third diameter different than the first diameter;
a fourth wire extending at least partially between the first end and the second end, the fourth wire having a fourth diameter different than the second diameter;
a thermistor connected to the first wire; and
a fixture protruding from an internal surface of the tube into a flow path of the supply of pressurized breathable air flowing through the tube, the thermistor being enclosed within the fixture.

35. The air delivery conduit of claim 34, further comprising a cuff coupled to the second end, the cuff including the internal surface, and the fixture protruding from the cuff.

36. The air delivery conduit of claim 34, wherein the first wire and the fourth wire form a sensing circuit, and the second wire and the third wire form a heating circuit distinct from the sensing circuit.

37. The air delivery conduit of claim 34, wherein the third wire and the fourth wire are connected to ground.

38. The air delivery conduit of claim 34, wherein the first diameter is equal to the fourth diameter.

39. The air delivery conduit of claim 34, wherein the second diameter is equal to the third diameter.

40. The air delivery conduit of claim 34, wherein the first diameter is equivalent to a gauge of 29 American Wire Gauge (AWG).

41. The air delivery conduit of claim 34, wherein the second diameter is equivalent to a gauge of 31 AWG.
